(19) United States
(12) Patent Application Publication
(10) Pub. No.: US 2025/0114636 A1
(43) Pub. Date: Apr. 10, 2025

(54) EXTERNAL FONTANELLE INTRACRANIAL LIGHT (EFIL) DEVICE

(71) Applicant: RUTGERS, THE STATE UNIVERSITY OF NEW JERSEY, New Brunswick, NJ (US)

(72) Inventor: Garrett Gianneschi, New Brunswick, NJ (US)

(21) Appl. No.: 18/903,209

(22) Filed: Oct. 1, 2024

Related U.S. Application Data
(60) Provisional application No. 63/542,429, filed on Oct. 4, 2023.

(51) Int. Cl. A61N 5/067; A61N 5/06

(52) U.S. Cl. CPC A61N 5/067; A61N 5/0613; A61N 2005/0626; A61N 2005/0632; A61N 2005/0643; A61N 2005/0652; A61N 2005/0653; A61N 2005/0663

(57) ABSTRACT

A device for treating neonatal intraventricular hemorrhage or brain injury comprises at least one laser configured to emit laser energy directed to a fontanelle or unfused suture of a neonatal cranium. The device includes a control unit electronically coupled to the laser or array of lasers. The control unit is configured to determine at least one laser parameter, such as a wavelength, an irradiance intensity, a voltage intensity, a power output, a focus angle, a focus distance, a duration of emission, a mode of emission, and a pulse frequency. A method of treatment using the device is also disclosed.

### REFERENCE TO RELATED APPLICATIONS

This application claims priority to U.S. Provisional Application No. 63/542,429 that was filed on Oct. 4, 2023. The entire content of the application referenced above is hereby incorporated by reference herein.

### FIELD

The present invention generally relates to External Fontanelle Intracranial Light (EFIL) devices, and, more particularly, to an EFIL device for the treatment of neonatal and infant brain injuries.

### BACKGROUND

Intraventricular hemorrhage (IVH) of the newborn is bleeding into the fluid-filled areas, or ventricles, surrounded by the brain. There is a high risk of IVH in premature infants because blood vessels in the brain are not yet fully developed and are extremely fragile. IVH typically occurs within the first several days of life. IVH is diagnosed via routine head ultrasound, which is recommended for all babies born before 30 weeks. Symptoms such as low blood count, apnea, and other signs of bleeding problems signal a need for diagnostic tests. There is no current therapy to stop the bleeding in premature infants with IVH. Symptomatic treatment, such as a blood transfusion, may be given to improve blood pressure and blood count. If hydrocephalus develops, a spinal tap or surgery may be required to relieve pressure and/or drain fluid from the brain.

Transcranial Photo Biomodulation (tPBM) is the use of light, directed at the head, to produce a bodily effect. It is believed that light therapy (e.g., using low-level lasers) causes the activation of transcription factors within cells, leading to the expression of many protective, anti-poptotic, anti-oxidant, and pro-proliferation gene products.¹ tPBM technology is currently being used to treat brain injury in adults. For example, NeuroThera™ and other red/near infrared light devices, directed at the head, have been shown to promote recovery, above a control group, in many studies of adults suffering from stroke or traumatic brain injury. The largest of these studies are the NEST-1 and NEST-2 trials (n=780), and pooled data showed clinically significant recovery of stroke patients over their control counterparts using the NIH stroke scale.^{2,3,4} Case reports of adults with traumatic brain injury have also shown tPBM to result in improved motor, cognitive, and sensory function.^{5, 7}

Studies have shown that photo biomodulation using 670 nm red light is safe in premature neonates of 24-26 weeks gestation.⁷ In addition, studies have shown that IVH-induced mice have better functional outcomes and decreased intracranial pressure with the used of red-light therapy compared to a control.⁸

Although research has shown that penetration of light therapy is limited through the skull, studies have been conducted to evaluate the safety and effectiveness of light therapy delivered to human soft tissue. Some models suggest that 808 nm wavelength light can penetrate up to 5 cm in human soft tissue.⁹ Other studies suggest that pulsing the light source can decrease the dose of light delivered to the skin surface, allow for greater dissipation of heat byproduct, and increase the power at a given depth of penetration.¹⁰

There is a need for a medical device that can safely and effectively provide intracranial light therapy to premature infants and neonates for the treatment of IVH and other brain injuries.

### BRIEF SUMMARY

A device for treating neonatal intraventricular hemorrhage or brain injury comprises at least one laser configured to emit laser energy directed to a fontanelle or unfused suture of a neonatal cranium. The device includes a control unit electronically coupled to the laser or array of lasers. The control unit is configured to determine at least one laser parameter, such as a wavelength, an irradiance intensity, a voltage intensity, a power output, a focus angle, a focus distance, a duration of emission, a mode of emission, and a pulse frequency. A method of treatment using the device is also disclosed.

Accordingly, certain embodiments provide a device for treating neonatal intraventricular hemorrhage or brain injury, the device comprising: at least one laser configured to emit laser energy directed to a fontanelle or unfused suture of a neonatal cranium; and a control unit electronically coupled to the at least one laser, the control unit configured to determine at least one laser parameter; wherein the at least one laser parameter is selected from the group consisting of a wavelength, an irradiance intensity, a voltage intensity, a power output, a focus angle, a focus distance, a duration of emission, a mode of emission, and a pulse frequency.

In certain embodiments, the device further comprises a platform configured to hold and position a neonate during treatment.

In certain embodiments, the platform comprises a cranial holder configured to expose and secure the neonatal cranium to allow laser energy to reach the fontanelle or unfused suture.

In certain embodiments, the at least one laser comprises a laser selected from the group consisting of: Light-Emitting Diodes (LEDs), Vertical-Cavity Surface-Emitting Lasers (VCSELs), Quantum Cascade Lasers (QCLs), Semiconductor Lasers, Solid-State Lasers, Fiber Lasers, Diode-Pumped Solid-State Lasers (DPSSLs), Optical Amplifiers, and Organic Light-Emitting Diodes (OLEDs).

In certain embodiments, the laser energy is directed through a neonate's Anterior Fontanelle, Posterior Fontanelle, or Sagittal Suture.

In certain embodiments, the control unit is configured to store the at least one laser parameter.

In certain embodiments, the control unit includes a display and user interface for monitoring and adjusting the at least one laser parameter.

In certain embodiments, the device comprises twelve lasers arranged in a circle or an arc.

In certain embodiments, the device comprises a first set of lasers and a second set of lasers, wherein the control unit is configured to operate the first and second sets of lasers in a Single Pole Double Throw (SPDT) relay.

In certain embodiments, the pulse frequency of the SPDT relay is 10 Hz.

In certain embodiments, the wavelength of the laser energy is about 650 nm to about 660 nm.

In certain embodiments, the control unit is electronically coupled to at least one physiological monitoring device configured to monitor a physiological parameter; and the control unit is configured to adjust the at least one laser parameter in response to the physiological parameter.

In certain embodiments, the physiological parameter comprises a parameter selected from the group consisting of: cerebral oxygenation, blood pressure, heart rate, temperature, electrocardiogram (ECG), respiratory rate (RR), oxygen saturation (SpO2), end-tidal CO2 (ETCO2), cardiac output (CO), central venous pressure (CVP), intracranial pressure (ICP), arterial blood gas (ABG), non-invasive cardiac monitoring (NICOM), pulse pressure, and mean arterial pressure (MAP).

Certain embodiments provide a method of treating neonatal intraventricular hemorrhage or brain injury, the method comprising: determining at least one laser parameter selected from the group consisting of a wavelength, an irradiance intensity, a voltage intensity, a power output, a focus angle, a focus distance, a duration of emission, a mode of emission, and a pulse frequency; and exposing a fontanelle or unfused suture of a neonatal cranium to laser energy characterized by the at least one laser parameter.

In certain embodiments, the method further comprises positioning the neonate on a platform prior to exposing the fontanelle or unfused suture of the neonatal cranium to laser energy.

In certain embodiments, the laser energy is directed to the neonate's Anterior Fontanelle, Posterior Fontanelle, or Sagittal Suture.

In certain embodiments, the method further comprises monitoring at least one physiological parameter and optionally adjusting the at least one laser parameter based on the at least one physiological parameter.

In certain embodiments, exposing the fontanelle or unfused suture of the neonatal cranium to laser energy comprises applying a SPDT relay at a frequency of 10 Hz.

In certain embodiments, the wavelength of the laser energy is about 650 nm to about 660 nm.

In certain embodiments, the duration of exposure is about one second to about 60 minutes.

This summary is intended to provide an overview of subject matter of the present patent application. It is not intended to provide an exclusive or exhaustive explanation of the invention. Other objects, features, and advantages of the present invention will be apparent to one of skill in the art from the following detailed description and figures.

### BRIEF DESCRIPTION OF THE FIGURES

In the drawings, which are not necessarily drawn to scale, like numerals may describe similar components in different views. Like numerals having different letter suffixes or subscripts may represent different instances of similar components. The drawings illustrate generally, by way of example, but not by way of limitation, various embodiments discussed in the present document.

FIG. 1 is a perspective view of an exemplary embodiment of an EFIL device in accordance with the present disclosure.

FIG. 2 includes various exemplary diagrammatic views of potential laser beam target areas on the neonatal cranium.

FIG. 3 is an enlarged view of characteristic fontanelles and sutures in the typical neonatal cranium.

FIG. 4A is a schematic drawing of a normal neonatal cranium showing the brain ventricles.

FIG. 4B is a schematic drawing of a neonatal cranium showing Grade III IVH and exemplary therapeutic laser placements.

FIG. 4C is a schematic drawing of a neonatal cranium showing Grade IV IVH and exemplary therapeutic laser placements.

### DETAILED DESCRIPTION

In the following description, for purposes of explanation, various details are set forth in order to provide a thorough understanding of some example embodiments. It will be apparent, however, to one skilled in the art that the present subject matter may be practiced without these specific details, or with slight alterations.

The anatomy of the neonatal cranium affords an opportunity for light therapy to be even more effective than in adults. In adults, the cranium is fused along suture lines, lacks fontanelles, and has thicker bones, which greatly limits irradiance penetration. However, the presence of fontanelles and unfused sutures in neonates and infants can allow greater light penetration into deep brain areas, such as the ventricles where blood pools as a result of IVH.

The present invention relates to an EFIL device designed to deliver targeted low-level laser therapy to neonates with IVH and other brain injuries or diseases. The device is particularly configured to direct therapeutic laser energy through the neonate's fontanelles and/or sutures, enabling non-invasive treatment aimed at reducing the severity of hemorrhage and promoting healing.

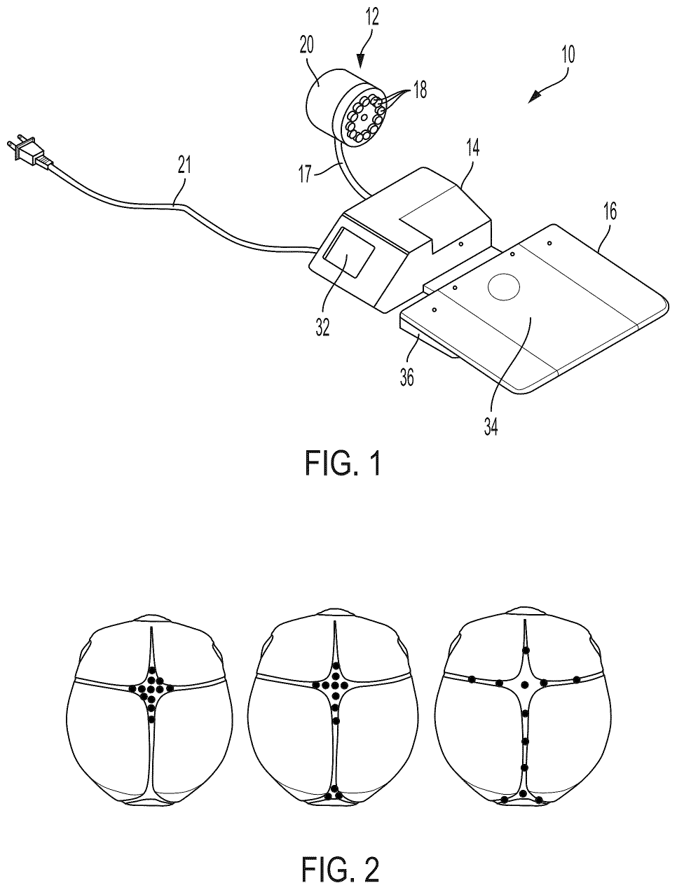

Referring now to the drawings wherein like reference numerals are used to identify like elements in the various views, FIG. 1 shows an embodiment of an EFIL device 10 configured to directly promote the resorption of IVH and heal pathologic brain tissue. The device 10 comprises a light column 12 and control unit 14. The device 10 can optionally include an infant platform 16, as shown in FIG. 1. The light column 12 can be reversibly coupled to the control unit 14, such as via an adjustable mount 17. The platform 16 can be reversibly coupled to the control unit 14 and/or light column 12 via the device housing or other suitable connection means. The device housing and platform 16 can be made from polylactic acid (PLA) or other non-toxic biodegradable or thermoplastic materials with comparable mechanical and chemical properties, including but not limited to Polyethylene Terephthalate Glycol-Modified (PETG), Polycaprolactone (PCL), Polyamide (Nylon), Polyhydroxyalkanoates (PHA), Thermoplastic Elastomers/Polyurethane (TPE/TPU), High-Density Polyethylene (HDPE), Polypropylene (PP), Acrylonitrile Butadiene Styrene-food-safe (ABS), and Polyhydroxybutyrate-co-valerate (PHBV).

The light column 12 comprises an array of lasers 18 positioned in an arc or circle and attached to laser housing 20. In the embodiment shown, there are twelve lasers 18 arranged in a circle; however, other embodiments can include fewer or more than twelve lasers 18 (e.g., between one and twenty lasers). The purpose of the arc or circular arrangement of the lasers 18 and dimensions of the light column 12 is to provide adequate vantage for the mounted laser diodes to illuminate the fontanelles and sutures of the patient. The lasers 18 can be mounted to the light column 12 in fixed or movable positions. In an exemplary embodiment, the lasers 18 can be mounted to the light column 12 with ball-and-socket joints, allowing for adjustability of laser distance and focus to address anatomic variations of neonatal craniums. The lasers 18 can be supplied by a power source (not shown), such as a 12V wall charger, via an electrical cable 21 coupled to circuitry within the control unit 14 and to the lasers 18. The laser source can include various types of laser diodes, such as Light-Emitting Diodes (LEDs), Vertical-Cavity Surface-Emitting Lasers (VCSELs), Quantum Cascade Lasers (QCLs), Semiconductor Lasers, Solid-State Lasers, Fiber Lasers, Diode-Pumped Solid-State Lasers (DPSSLs), Optical Amplifiers, Organic Light-Emitting Diodes (OLEDs), depending on the desired output characteristics.

The EFIL device 10 can be operated in various modes, allowing for the adjustment of beam parameters such as intensity, focus, and pulse frequency. The control unit 14 can be configured to store pre-set treatment protocols or allow manual adjustment by the clinician. For example, the lasers 18 can emit laser beams at wavelengths ranging from about 450 nm to about 1000 nm. In an embodiment, laser beams at wavelengths of about 650 nm to about 660 nm are emitted. The power output of the laser beams can range from about 1 mW to about 250 mW per beam. In an embodiment, the width of a single laser beam can be about 1.5 mm at the light column source. The light column 12 can be configured to provide irradiance upon a neonate's cranial epidermis of greater than 0 mW/cm² to no more than about 150 mW/cm² to prevent excessive heating. The light column 12 can be positioned about 5 cm to about 15 cm from the neonate's head to allow for maneuvering of the lasers 18 and dissipation of minimal heat generated within the lasers 18. In an embodiment, the lasers 18 can be configured to emit continuous or pulsed laser beams, with pulse durations adjustable between 1 millisecond and 1 second. In an embodiment, the laser array can pulse at about 10 Hz. The laser pulse frequency can be controlled by a microcontroller within the control unit 14, described further below.

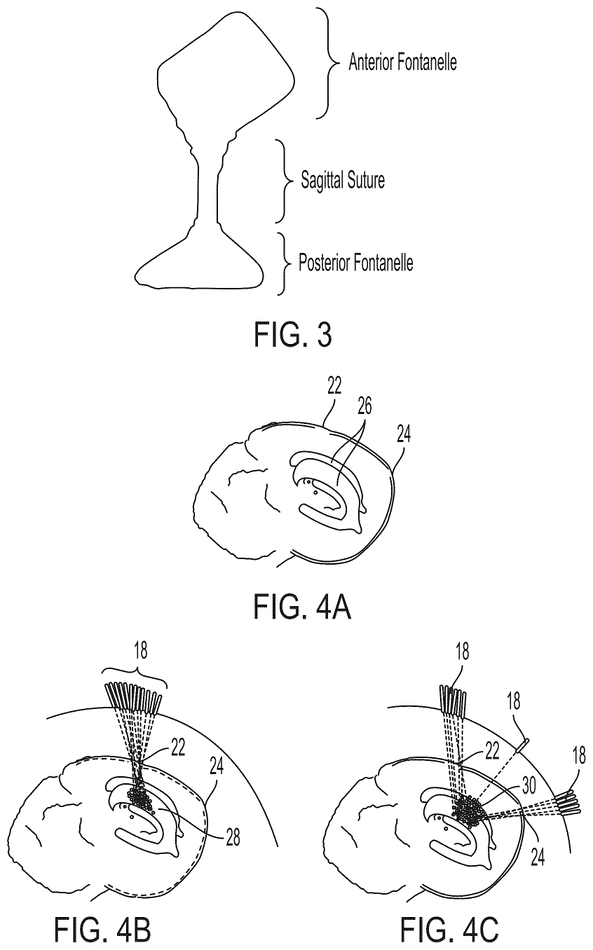

One or more laser beams can be directed through an infant's fontanelles and unfused sutures. Anatomically, the lasers 18 can be positioned superior to the neonatal head and aimed inferiorly. The angle of the individual lasers can optimize coverage based, for example, on a tissue scattering profile using Mie scattering and Monte Cristo calculations.^{2,3} FIG. 2 illustrates potential laser beam target areas, represented by dots on the neonatal cranium. The laser array can be arranged to emit laser energy through target areas are over the infant's Anterior Fontanelle, Sagittal Suture, and Posterior Fontanelle. FIG. 3 further illustrates the characteristic diamond shape of the Anterior Fontanelle, the triangle shape of the Posterior Fontanelle, and the Sagittal Suture connecting the two fontanelles.

As shown in FIGS. 4A-C, laser beams directed through one or more of the fontanelle or suture openings can travel toward the vascular insult and pathologic tissue within the brain ventricles. FIG. 4A depicts a normal neonatal cranium with an open anterior fontanelle region 22 and an open posterior fontanelle region 24. Normal brain ventricles 26 are shown without any indication of bleeding. FIG. 4B depicts Grade III IVH 28 with twelve lasers 18 emitting light that passes passing through the anterior fontanelle region 22. FIG. 4C depicts Grade IV IVH 30 with a variable laser set up, including six lasers 18 focused on the anterior fontanelle region 22, five lasers 18 focused on the posterior fontanelle region 24, and one laser 18 focused on the sagittal suture region (not shown).

Returning now to FIG. 1, the control unit 14 includes a microcontroller with device circuitry (not shown) and a power supply (not shown) electrically coupled to a power source via cable 21. In an embodiment, the main power source is a standard US wall socket of 120V and 15 A of DC. The wall socket powers both the microcontroller and the light column 12. The microcontroller uses between about 5V and about 200 mA. The light column 12 uses a variable resistor (rheostat) to provide between about 3-5V and about 1.2 A. The microcontroller can operate a Single Pole Double Throw (SPDT) relay, which switches at a rate of 10 Hz between two sections of the laser array (e.g., one section may be the six diodes forming the left side of the circular laser array and another section may be the six diodes forming the right side of the circular laser array). Both the microcontroller and light column circuit are connected to a negative pole or ground.

The control unit 14 further includes a display 32 including a user interface coupled to the microcontroller and allowing for the monitoring and adjustment of various parameters, including intensity, focus angle, duration, and frequency of the laser emission. For example, the display 32 can show intensity levels for both voltage and irradiance of the laser array. The irradiance produced at the infant's fontanelle is essentially the “dosage” of treatment, and it must be carefully monitored by the operating clinician. If the irradiance is too high, it may be harmful and cause thermal heating; and if it is too low, then it may have no therapeutic effect. The display 32 can include a time duration, which can be set between 0 and 60 minutes, for example, per treatment session. The duration is also a component of the “dosage” of the low-level laser therapy that the device delivers, with longer durations resulting in larger dosages. The display 32 can indicate the mode of laser emission (e.g., continuous or pulsed), and the user interface can be used to adjust modes, pulse frequency, and/or the SPDT relay. Furthermore, the microcontroller can be coupled to other physiological monitors, allowing the display 32 to show parameters such as cerebral oxygenation, blood pressure, heart rate, temperature, electrocardiogram (ECG) measuring the electrical conduction wave of the heart, capnogram measuring pressure differential of the breathing cycle as a continuous wave displayed on a monitor, respiratory rate (RR), oxygen saturation (SpO2), End-Tidal CO2 (ETCO2), Cardiac output (CO), Central Venous Pressure (CVP), Intracranial pressure (ICP), Arterial Blood Gas (ABG), non-invasive cardiac monitoring (NICOM), pulse pressure, mean arterial pressure (MAP). The microcontroller can be configured to adjust the wavelength, irradiance intensity, voltage intensity, power output, focus angle, focus distance, duration of emission, mode of emission, pulse frequency, and other laser parameters in response the physiological parameters.

The infant platform 16, also shown in FIG. 1, is intended to hold, expose and slightly elevate the infant cranium. The platform 16 can include a neonatal cranial holder 34 to help position the infant. The neonatal cranial holder 34 can be about 5 cm at its inner diameter with cushioning of about 1 cm width which extends the outer diameter to 7 cm. The cranial holder 34 is configured to expose and gently secure the infant cranium while allowing the lasers 18 access to the fontanelles. In an embodiment, the platform 16 can be about 20-60 cm long and about 25-30 cm wide, designed to fit into both a bassinet and an isolette. The platform itself is flat, but the cranial end of the platform can be raised about 5 cm via used of an incline 36 or the like attached to the bottom of the platform 16.

An exemplary embodiment of a method of use and operation of the EFIL device 10 for the treatment of IVH and other brain injuries will now be described. A patient in need of treatment, such as neonate that has been diagnosed with Grade III or IV IVH via ultrasound or equivalent imaging, is (ideally) wrapped and swaddled in a small blanket to induce sleep and avoid major movement. The EFIL device 10 is placed in the neonate's bassinet or isolette. The swaddled infant is then laid in the prone position in the center of the platform 16, with their head placed in the cushioned cranial holder 34 to elevate and more fully exposes the fontanelles and sutures. The light column 12 is adjusted as needed, such that the lasers 18 are able to provide about 360 degrees of vantage of the neonate's head. The device 10 can be plugged in to a wall socket with a 12V, 1.5 A DC current and switched ON. The control unit 14 can be configured to activate half of the lasers (e.g., six diodes forming the left side of the circle) to pulse at 10 hz in alternation with the other half of the lasers (e.g., six diodes forming the right side of the circle). The direct current switches between running in the six diodes of the left-side circuit and running in the six diodes of the right-side side circuit. A rheostat within the control unit 14 allows for adjustable voltage from 3-5V, allowing for irradiance upon the skull between 0-150 mW/cm². The infant will (presumably) remain relatively still while the head is illuminated for the desired amount of time. The control unit 14 can be configured to adjust the duration of laser exposure from 1 second to 1 hour, typically ranging from 10 seconds to 10 minutes per treatment session. Once the treatment is finished, the device is switched to OFF and disconnected from the wall socket. The neonate is then removed from the device 10.

In one example, the device 10 is used to treat a neonate with Grade III IVH. The clinician selects a protocol that emits four laser beams at 810 nm with a power output of 50 mW per beam. The treatment lasts for 5 minutes, during which the device monitors the neonate's cerebral oxygenation and adjusts the beam intensity as needed.

In another example, the device 10 is configured to deliver a single 670 nm beam at 10 mW for a duration of 30 seconds, targeting a specific area of hemorrhage. Multiple different laser wavelengths can be used in conjunction with one another in series or simultaneously.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment,” or the like, means that a particular feature, structure, or characteristic described in connection with the embodiment is included in at least one embodiment. Thus, appearances of the phrases “in various embodiments,” “in some embodiments,” “in one embodiment,” or “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The terms “about” and “approximately” may be used throughout the specification when referring to a measurable value, such as an amount, a distance, a temporal duration, and the like. The terms “about” and “approximately” are meant to encompass variations of ±20% or ±10%, in certain embodiments ±5%, in certain embodiments ±1%, in certain embodiments ±0.1% from the specified value, as such variations are appropriate in accordance with the present disclosure.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

### CITATIONS

1. Hashmi JT, Huang YY, Osmani BZ, Sharma SK, Naeser MA, Hamblin MR. Role of low-level laser therapy in neurorehabilitation. PM R. 2010 December;2(12 Suppl 2):S292-305. doi: 10.1016/j.pmrj.2010.10.013. PMID: 21172691; PMCID: PMC3065857.
2. Lampl Y, Zivin JA, Fisher M, Lew R, Welin L, Dahlof B, Borenstein P, Andersson B, Perez J, Caparo C, Ilic S, Oron U. Infrared laser therapy for ischemic stroke: a new treatment strategy: results of the NeuroThera Effectiveness and Safety Trial-1 (NEST-1). Stroke. 2007 June;38(6):1843-9. doi: 10.1161/STROKEAHA.106.478230. Epub 2007 Apr. 26. PMID: 17463313.
3. Zivin, J.A., G.W. Albers, N. Bornstein, T. Chippendale, B. Dahlof, T. Devlin, M. Fisher, W. Hacke, W. Holt, S. Ilic, S. Kasner, R. Lew, M. Nash, J. Perez, M. Rymer, P. Schellinger, D. Schneider, S. Schwab, R. Veltkamp, M.Walker, and J. Streeter, Effectiveness and safety of transcranial laser therapy for acute ischemic stroke. Stroke, 2009. 40(4): pp. 1359-1364.
4. Huisa, Branko N., et al. “Transcranial laser therapy for acute ischemic stroke: a pooled analysis of NEST-1 and NEST-2.” International Journal of Stroke 8.5 (2013): 315-320.
5. Naeser MA, Saltmarche A, Krengel MH, Hamblin MR, Knight JA. Improved cognitive function after transcranial, light-emitting diode treatments in chronic, traumatic brain injury: two case reports. Photomed Laser Surg. 2011 May;29(5):351-8. doi: 10.1089/pho.2010.2814. Epub 2010 Dec. 23. PMID: 21182447; PMCID: PMC3104287.
6. Naeser MA, Zafonte R, Krengel MH, et al. Significant improvements in cognitive performance post-transcranial, red/near-infrared light-emitting diode treatments in chronic, mild traumatic brain injury: open-protocol study. J Neurotrauma. 2014;31(11):1008-1017. doi:10.1089/neu.2013.3244.
7. Kent AL, Broom M, Parr V, Essex RW, Abdel-Latif ME, Dahlstrom JE, Valter K, Provis J, Natoli R. A safety and feasibility study of the use of 670 nm red light in premature neonates. J Perinatol. 2015 July;35(7):493-6. doi: 10.1038/jp.2015.5. Epub 2015 Feb. 19. PMID: 25695843.
8. Dong-Yu Li, Shao-Jun Liu, Ting-Ting Yu, Zhang Liu, Si-Lin Sun, Denis Bragin, Nikita Navolokin, Jürgen Kurths, Oxana Glushkovskaya-Semyachkina, Dan Zhu Photostimulation of lymphatic clearance of red blood cells from the mouse brain after intraventricular hemorrhage. bioRxiv 2020.11.16.384149; doi: https://doi.org/10.1101/2020.11.16.384149.
9. Esnouf A, Wright PA, Moore JC, Ahmed S. Depth of penetration of an 850 nm wavelength low level laser in human skin. AcupunctElectrother Res. 2007; 32(1-2):81-6.
10. Henderson TA, Morries LD. Near-infrared photonic energy penetration: can infrared phototherapy effectively reach the human brain? Neuropsychiatr Dis Treat. 2015 Aug. 21;11:2191-208. doi: 10.2147/NDT.S78182. PMID: 26346298; PMCID: PMC4552256.

What is claimed is:

1. A device for treating neonatal intraventricular hemorrhage or brain injury, the device comprising:
at least one laser configured to emit laser energy directed to a fontanelle or unfused suture of a neonatal cranium; and
a control unit electronically coupled to the at least one laser, the control unit configured to determine at least one laser parameter;
wherein the at least one laser parameter is selected from the group consisting of a wavelength, an irradiance intensity, a voltage intensity, a power output, a focus angle, a focus distance, a duration of emission, a mode of emission, and a pulse frequency.

2. The device of claim 1, further comprising a platform configured to hold and position a neonate during treatment.

3. The device of claim 2, wherein the platform comprises a cranial holder configured to expose and secure the neonatal cranium to allow laser energy to reach the fontanelle or unfused suture.

4. The device of claim 1, wherein the at least one laser comprises a laser selected from the group consisting of: Light-Emitting Diodes (LEDs), Vertical-Cavity Surface-Emitting Lasers (VCSELs), Quantum Cascade Lasers (QCLs), Semiconductor Lasers, Solid-State Lasers, Fiber Lasers, Diode-Pumped Solid-State Lasers (DPSSLs), Optical Amplifiers, and Organic Light-Emitting Diodes (OLEDs).

5. The device of claim 1, wherein the laser energy is directed through a neonate's Anterior Fontanelle, Posterior Fontanelle, or Sagittal Suture.

6. The device of claim 1, wherein the control unit is configured to store the at least one laser parameter.

7. The device of claim 1, wherein the control unit includes a display and user interface for monitoring and adjusting the at least one laser parameter.

8. The device of claim 1, comprising twelve lasers arranged in a circle or an arc.

9. The device of claim 1, comprising a first set of lasers and a second set of lasers, wherein the control unit is configured to operate the first and second sets of lasers in a Single Pole Double Throw (SPDT) relay.

10. The device of claim 9, wherein the pulse frequency of the SPDT relay is 10 Hz.

11. The device of claim 1, wherein the wavelength of the laser energy is about 650 nm to about 660 nm.

12. The device of claim 1, wherein the control unit is electronically coupled to at least one physiological monitoring device configured to monitor a physiological parameter; and wherein the control unit is configured to adjust the at least one laser parameter in response to the physiological parameter.

13. The device of claim 12, wherein the physiological parameter comprises a parameter selected from the group consisting of: cerebral oxygenation, blood pressure, heart rate, temperature, electrocardiogram (ECG), respiratory rate (RR), oxygen saturation (SpO2), end-tidal CO2 (ETCO2), cardiac output (CO), central venous pressure (CVP), intracranial pressure (ICP), arterial blood gas (ABG), non-invasive cardiac monitoring (NICOM), pulse pressure, and mean arterial pressure (MAP).

14. A method of treating neonatal intraventricular hemorrhage or brain injury, the method comprising:
determining at least one laser parameter selected from the group consisting of a wavelength, an irradiance intensity, a voltage intensity, a power output, a focus angle, a focus distance, a duration of emission, a mode of emission, and a pulse frequency; and
exposing a fontanelle or unfused suture of a neonatal cranium to laser energy characterized by the at least one laser parameter.

15. The method of claim 14, further comprising positioning the neonate on a platform prior to exposing the fontanelle or unfused suture of the neonatal cranium to laser energy.

16. The method of claim 14, wherein the laser energy is directed to the neonate's Anterior Fontanelle, Posterior Fontanelle, or Sagittal Suture.

17. The method of claim 14, further comprising monitoring at least one physiological parameter and optionally adjusting the at least one laser parameter based on the at least one physiological parameter.

18. The method of claim 14, wherein exposing the fontanelle or unfused suture of the neonatal cranium to laser energy comprises applying a SPDT relay at a frequency of 10 Hz.

19. The method of claim 14, wherein the wavelength of the laser energy is about 650 nm to about 660 nm.

20. The method of claim 14, wherein the duration of exposure is about one second to about 60 minutes.
